(19) United States
(12) Patent Application Publication
(10) Pub. No.: US 2025/0152495 A1
(43) Pub. Date: May 15, 2025

(54) COMPOSITION COMPRISING A PHOTOCROSSLINKABLE POLYMER HAVING A HYDROPHOBIC GROUP AND A COLORING AGENT

(71) Applicant: L'OREAL, Paris (FR)

(72) Inventors: Eric PARRIS, SAINT-OUEN (FR); Gabin VIC, SAINT-OUEN (FR); Frédéric WOODLAND, SAINT-OUEN (FR)

(21) Appl. No.: 18/722,395

(22) PCT Filed: Dec. 16, 2022
(86) PCT No.: PCT/EP2022/086465

§ 371 Date: Jun. 20, 2024

(30) Foreign Application Priority Data
Dec. 23, 2021 (FR) FR2114345

(51) Int. Cl. A61K 8/81; A61Q 5/06

(52) U.S. Cl. CPC A61K 8/8135; A61Q 5/065; A61K 2800/43; A61K 2800/81; A61K 2800/95

(57) ABSTRACT

The present invention relates to a composition (C) for the treatment of keratin fibers, comprising at least one photocrosslinkable polymer having hydrophobic groups, and at least one coloring agent chosen from pigments and mixtures thereof.

The present invention also relates to a cosmetic treatment process, in particular for dyeing keratin fibers, wherein the composition (C) is applied to said fibers.

A subject of the present invention is a composition (C) for the treatment of keratin fibers, comprising at least one photocrosslinkable polymer, and at least one coloring agent chosen from pigments and mixtures thereof.

The present invention also relates to a process for treating keratin fibers.

### TECHNICAL FIELD

In the field of dyeing of keratin fibers, in particular human keratin fibers, it is already known practice to dye keratin fibers via various techniques starting from direct dyes or pigments for non-permanent dyeing or from dye precursors for permanent dyeing.

There are essentially three types of process for coloring the hair:
a) “permanent” coloring, the function of which is to afford a substantial modification to the natural color and which uses oxidation dyes which penetrate into the hair fiber and forms the dye via an oxidative condensation process;
b) non-permanent, semi-permanent or direct dyeing, which does not employ the oxidative condensation process and withstands 4 or 5 shampoo washes; it consists in dyeing keratin fibers with dye compositions containing direct dyes;
c) temporary dyeing, which gives rise to a modification to the natural color of the head of hair which remains from one shampooing operation to the next, and which serves to enhance or correct a shade which has already been obtained. It may also be likened to a “makeup” process.

For this last type of coloring, it is known practice to use colored polymers formed by grafting one or more dyes of azo, triphenylmethane, azine, indoamine or anthraquinone nature onto a polymer chain. These colored polymers are not entirely satisfactory, notably as regards the homogeneity of the coloring obtained and its resistance, not to mention the problems associated with their manufacture and notably with their reproducibility.

Another coloring method consists in using pigments. Specifically, the use of pigment on the surface of keratin fibers generally makes it possible to obtain colorings that are visible on dark hair, since the surface pigment masks the natural color of the fiber. However, the colorings obtained via this coloring method have the drawback of having poor resistance to shampoo washing and also to external agents such as sebum, perspiration, brushing and/or rubbing.

In addition, compositions for temporarily dyeing the hair may also lead to a hair feel that is uncosmetic and/or not natural; the hair thus dyed may notably lack softness and/or suppleness and/or strand separation.

In addition, there are no effective makeup-removing compositions for removing this type of temporary dye composition when it is persistent with respect to shampoo washing.

The need thus remains for a composition for treating keratin fibers, notably the hair, which has the advantage of obtaining a homogeneous and smooth colored coating on the hair, and also hair with complete strand separation, while at the same time forming a coating that is persistent with respect to shampoo washing and to the various attacking factors to which the hair may be subjected, such as blow-drying and/or rubbing, without degrading the hair. There is also a need to be able to eliminate this colored coating when so desired.

Thus, the aim of the present invention is to develop a composition for treating keratin fibers, notably the hair, which has the advantage of obtaining a homogeneous and smooth colored coating on the hair, and also hair with complete strand separation, while at the same time forming a coating that is persistent with respect to shampoo washing and to the various attacking factors to which the hair may be subjected, such as blow-drying and/or rubbing, without degrading the hair. Advantageously, the colored coating can be readily eliminated when so desired. Indeed, the coating according to the invention does not lead to chemical modification of the keratin fibers, unlike the case with grafting on to said fibers. The fiber integrity is respected.

### DISCLOSURE OF THE INVENTION

A subject of the invention is thus a cosmetic composition (C) for the treatment of keratin fibers, comprising:
a) at least one photocrosslinkable polymer comprising at least one photodimerizable group and at least one hydrophobic group, and
b) at least one coloring agent chosen from pigments and mixtures thereof,
the hydrophobic group(s) being chosen from:
   a saturated or unsaturated (C₁-C₃₀)alkyl group, optionally substituted and/or interrupted with one or more heteroatoms,
   an alkenyl group,
   a fluorinated group such as a fluorocarbon group such as —CF₃, —CHF₂, —OCF₃, —SCF₃, CF₃C(O)—,
   a silicone group such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, with Rₐ, R_{b} and R_{c}, which may be identical or different, representing a (C₁-C₃)alkyl group which is optionally interrupted and/or terminated with one or more non-contiguous heteroatoms such as O or S, and R representing a (C₁-C₆)alkyl group.

The present invention also relates to a process for the cosmetic treatment, in particular the dyeing, of keratin fibers such as the hair, wherein the composition (C) as defined above is applied to said fibers.

Accordingly, another subject of the invention is a process for treating keratin materials comprising the application of a composition comprising at least one photocrosslinkable polymer comprising at least one pendent photodimerizable group and at least one hydrophobic pendent group and a step of irradiating the composition on the keratin materials in order to crosslink the composition.

Via the use of this composition (C) on keratin fibers, colored coatings are obtained on the hair that make it possible to obtain a coloring that is visible on all types of hair in a manner that is persistent with respect to shampoo washing, while at the same time preserving the physical qualities of the keratin fibers. Such a coating may be resistant to the external attacking factors to which the hair may be subjected, such as blow-drying and perspiration. It makes it possible in particular to obtain a smooth and uniform deposit.

Moreover, this composition makes it possible to obtain hair with perfect strand separation, which can be styled without problem and which has good cosmetic properties, notably in terms of softness and feel.

Advantageously, the colored coating thus obtained can be readily eliminated by means of a makeup-removing composition.

The term “hair with strand separation” means hair which, after application of the composition and drying, is not stuck together (or of which all the strands are separated from each other) and thus does not form clumps of hair.

For the purposes of the present invention, “coloring which is persistent with regard to shampooing operations” is understood to mean that the coloring obtained persists after one shampooing operation, preferably after three shampooing operations, more preferentially after five shampooing operations.

The expression “keratin fibers” is understood particularly to mean human keratin fibers, such as the hair, eyelashes, eyebrows and body hair, preferentially the hair, eyebrows and eyelashes, more preferentially still the hair.

The term “at least one” means one or more.

For the purposes of the invention, the term “polymer” means a compound corresponding to the repetition of one or more units (these units being derived from compounds known as monomers). This or these unit(s) are repeated at least twice and preferably at least three times.

The invention is not limited to the illustrated examples. The characteristics of the various examples may notably be combined within variants which are not illustrated.

For the purposes of the present invention and unless otherwise indicated:
an “alkylene chain” represents an acyclic C₁-C₂₀ divalent hydrocarbon chain, particularly a C₁-C₆ chain, more particularly a C₁-C₂ chain when the chain is linear, optionally substituted with one or more groups, which may be identical or different, chosen from i) hydroxyl, ii) (C₁-C₂)alkoxy, iii) (poly)hydroxy(C₂-C₄)alkoxy(di)(C₁-C₂)(alkyl)amino, iv) R^{a}—Z^{a}—C(Z^{b})—Z^{c}—, and v) R^{a}—Z^{a}—S(O)ₜ—Z^{c}— with Z^{a} and Z^{b}, which may be identical or different, representing an oxygen or sulfur atom, or a group NR^{a′}, Z^{c}, representing a bond, an oxygen or sulfur atom, or a group NR^{a}; R^{a}, representing an alkali metal, a hydrogen atom, an alkyl group, or alternatively is absent if another part of the cationic molecule and R^{a′} representing a hydrogen atom or an alkyl group; more particularly, the groups iv) are chosen from carboxylate —C(O)O⁻ or —C(O)OMetal (Metal=alkali metal), carboxyl —C(O)—OH, guanidino H₂H—C(NH₂)—NH—, amidino H₂H—C(NH₂)—, (thio)ureo H₂N—C(O)—NH— and H₂N—C(S)—NH—, aminocarbonyl —C(O)—NRa′₂ or aminothiocarbonyl —C(S)—NRa′₂; carbamoyl Ra′—C(O)—NRa′— or thiocarbamoyl Ra′—C(S)—NRa′— with Ra′, which may be identical or different, representing a hydrogen atom or a (C₁-C₄)alkyl group;
the “aryl” or “heteroaryl” radicals or the aryl or heteroaryl part of a radical may be substituted with at least one substituent borne by a carbon atom, chosen from:
   a C₁-C₁₆ and preferably C₁-C₈ alkyl radical, optionally substituted with one or more radicals chosen from the radicals hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom;
   a hydroxyl group;
   a C₁-C₂ alkoxy radical;
   a C₂-C₄ (poly)hydroxyalkoxy radical;
   an amino radical;
   a 5- or 6-membered heterocycloalkyl radical;
   an optionally cationic 5- or 6-membered heteroaryl radical, preferentially imidazolium, optionally substituted with a (C₁-C₄)alkyl radical, preferentially methyl;
an amino radical substituted with one or two identical or different C₁-C₆ alkyl radicals, optionally bearing at least:
i) a hydroxyl group,
ii) an amino group optionally substituted with one or two optionally substituted C₁-C₃ alkyl radicals, said alkyl radicals possibly forming, with the nitrogen atom to which they are attached, a saturated or unsaturated, optionally substituted 5- to 7-membered heterocycle, optionally comprising at least one other nitrogen or non-nitrogen heteroatom,
iii) one quaternary ammonium group —N⁺R′R″R′″, M⁻ for which R′, R″ and R′″, which may be identical or different, represent a hydrogen atom or a C₁-C₄ alkyl group; and M⁻ represents the counterion of the organic or mineral acid or of the corresponding halide;
iv) or an optionally cationic 5- or 6-membered heteroaryl radical, preferentially imidazolium, optionally substituted with a (C₁-C₄)alkyl radical, preferentially methyl;
   an acylamino radical (—NR—C(O)—R′) wherein the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) wherein the radicals R, which may be identical or different, represent a hydrogen atom or a C₁-C₄ alkyl radical optionally bearing at least one hydroxyl group;
   an alkylsulfonylamino radical (R′—S(O)₂—N(R)—) wherein the R radical represents a hydrogen atom or a C₁-C₄ alkyl radical optionally bearing at least one hydroxyl group and the R′ radical represents a C₁-C₄ alkyl radical or a phenyl radical; an aminosulfonyl radical ((R)₂N—S(O)₂—) wherein the R radicals, which may be identical or different, represent a hydrogen atom or a C₁-C₄ alkyl radical optionally bearing at least one hydroxyl group,
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
the cyclic, cycloalkyl or heterocyclic part of a non-aromatic radical may be substituted with at least one substituent chosen from the following groups:
   hydroxyl;
   C₁-C₄ alkoxy or C₂-C₄ (poly)hydroxyalkoxy;
   C₁-C₄ alkyl;
   alkylcarbonylamino (R—C(O)—N(R′)—) wherein the R′ radical is a hydrogen atom or a C₁-C₄ alkyl radical optionally bearing at least one hydroxyl group and the R radical is a C₁-C₂ alkyl radical or an amino radical optionally substituted with one or two identical or different C₁-C₄ alkyl groups, themselves optionally bearing at least one hydroxyl group, said alkyl radicals possibly forming, with the nitrogen atom to which they are attached, a saturated or unsaturated and optionally substituted 5- to 7-membered heterocycle, optionally comprising at least one other nitrogen or non-nitrogen heteroatom;
   alkylcarbonyloxy (R—C(O)—O—) wherein the R radical is a C₁-C₄ alkyl radical or an amino group optionally substituted with one or two identical or different C₁-C₄alkyl groups, themselves optionally bearing at least one hydroxyl group, where said alkyl radicals may form, with the nitrogen atom to which they are attached, a saturated or unsaturated and optionally substituted 5- to 7-membered heterocycle optionally comprising at least one other nitrogen or non-nitrogen heteroatom;
   alkoxycarbonyl (R-G-C(O)—) wherein the R radical is a C₁-C₄ alkoxy radical, G is an oxygen atom or an amino group optionally substituted with a C₁-C₄ alkyl group optionally bearing at least one hydroxyl group, where said alkyl radical may form with the nitrogen atom to which it is attached a saturated or unsaturated, optionally substituted 5- to 7-membered heterocycle, optionally comprising at least one other nitrogen or non-nitrogen heteroatom;
a cyclic, cycloalkyl or heterocyclic radical, or a non-aromatic part of an aryl or heteroaryl radical, which may also be substituted with one or more oxo groups;
a cycloalkyl radical is a mono- or bicyclic, hydrocarbon radical comprising 3 to 10 carbon atoms, preferably 4 to 7 carbon atoms, such as cyclopentyl or cyclohexyl;
a hydrocarbon-based chain is unsaturated when it includes one or more double bonds and/or one or more triple bonds;
an “aryl” radical represents a monocyclic or fused or non-fused polycyclic carbon-based group comprising from 6 to 22 carbon atoms, and wherein at least one ring is aromatic; preferentially, the aryl radical is a phenyl, biphenyl, naphthyl, indenyl, anthracenyl or tetrahydronaphthyl;
a “heteroaryl radical” represents an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
a “heterocyclic radical” is a 5- to 22-membered, monocyclic or fused or non-fused polycyclic radical that may contain one or two unsaturations but is not aromatic, including from 1 to 6 heteroatoms chosen from nitrogen, oxygen, sulfur and selenium;
a “heterocycloalkyl radical” is a heterocyclic radical comprising at least one saturated ring;
a “cationic heteroaryl radical” is a heteroaryl group as defined above that includes an endocyclic or exocyclic quaternized cationic group,
when the cationic charge is endocyclic, it is included in the electron delocalization via the mesomeric effect; for example, it is a pyridinium, imidazolium or indolinium group:

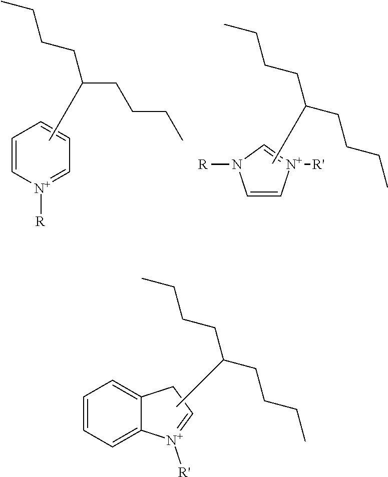

with R and R′ being a heteroaryl substituent as defined previously and particularly a (hydroxy)(C₁-C₃)alkyl group such as methyl;
when the cationic charge is exocyclic, it is, for example, an ammonium or phosphonium R⁺ substituent, such as trimethylammonium, which is outside the heteroaryl, such as pyridinyl, indolyl, imidazolyl or naphthalimidyl, in question:

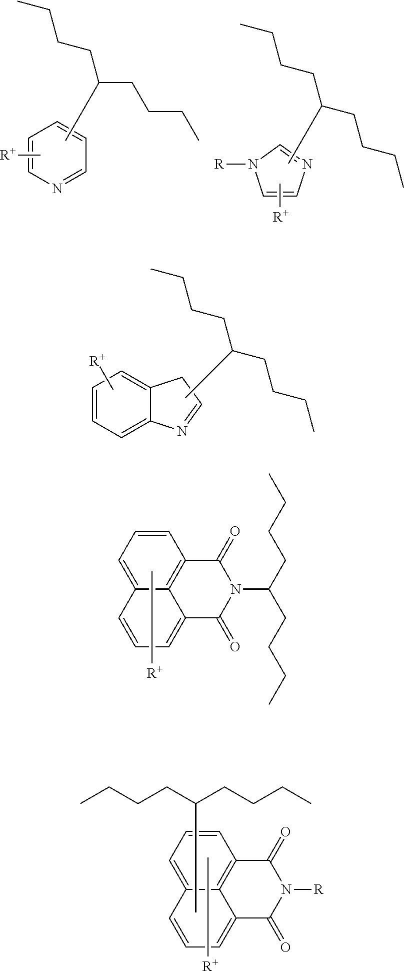

with R a heteroaryl substituent as defined above and R⁺ an ammonium RₐR_{b}R_{c}N⁺—, phosphonium RₐR_{b}R_{c}P⁺- or ammonium RₐR_{b}R_{c}N⁺—(C₁-C₆)alkylamino group with Rₐ, R_{b} and R_{c}, which may be identical or different, representing a hydrogen atom or a (C₁-C₃)alkyl group such as methyl;
a “cationic aryl bearing an exocyclic charge” means an aryl ring of which the quaternized cationic group is outside said ring; it is especially an ammonium or phosphonium R⁺ substituent, such as trimethylammonium, which is outside the aryl, such as phenyl or naphthyl:

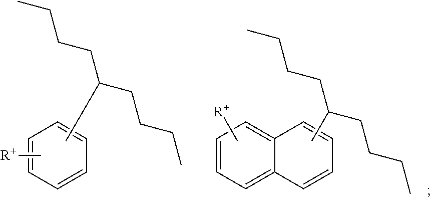

an “alkyl radical” is a linear or branched C₁-C₂₀ and preferably C₁—C hydrocarbon-based radical;
an “alkenylene radical” is an unsaturated hydrocarbon-based divalent radical as defined previously, which may contain from 1 to 4 conjugated or unconjugated double bonds —C═C—; the alkenylene group particularly contains 1 or 2 unsaturations;
the term “optionally substituted” applied to the alkyl radical means that said alkyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, where said alkyl radicals may form with the nitrogen atom that bears them a 5- to 7-membered heterocycle, optionally comprising one other nitrogen or non-nitrogen heteroatom; v) or a quaternary ammonium group —N⁺R′R″R′″, M⁻ for which R′, R″ and R′″, which may be identical or different, represent a hydrogen atom or a C₁-C₄ alkyl group, or else —N⁺R′R″R′″ forms a heteroaryl such as imidazolium optionally substituted with a C₁-C₄ alkyl group, and M⁻ represents the counterion of the corresponding organic acid, inorganic acid or halide;
an “alkoxy radical” is an alkyloxy radical for which the alkyl radical is a linear or branched C₁-C₁₆ and preferentially C₁-C₈ hydrocarbon-based radical; when the alkoxy group is optionally substituted, this implies that the alkyl group is optionally substituted as defined above;
the term “organic or mineral acid salt” more particularly means salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
The term “anionic counterion” is intended to mean an anion or an anionic group derived from an organic or mineral acid salt which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Alk-O—S(O)O⁻ such as benzene sulfate and toluene sulfate; xi) alkoxy sulfates: Alk-O—S(O)₂O— such as methoxy sulfate and ethoxy sulfate; xii) aryloxy sulfates: Ar—O—S(O)₂O⁻, xiii) phosphates O═P(OH)₂—O⁻, O=P(O⁻)₂—OH O=P(O⁻)₃, HO—[P(O)(O⁻)]_{w}—P(O)(O⁻)₂ with w being an integer; xiv) acetate; xv) triflate; and xvi) borates such as tetrafluoroborate, xvii) disulfate (O═)₂S(O⁻)₂ or SO₄²⁻ and monosulfate HSO₄⁻; the anionic counterion, derived from an organic or inorganic acid salt, ensures the electrical neutrality of the molecule; thus, it is understood that when the anion comprises several anionic charges, then the same anion may serve for the electrical neutrality of several cationic groups in the same molecule or else may serve for the electrical neutrality of several molecules; for example, polymers that comprise two cationic entities may contain either two “singly charged” anionic counterions or one “doubly charged” anionic counterion such as (O═)₂S(O⁻)₂ or O=P(O⁻)₂—OH;
moreover, the addition salts that may be used in the context of the invention are notably chosen from addition salts with a cosmetically acceptable base such as the basifying agents as defined below, for instance alkali metal hydroxides such as sodium hydroxide or potassium hydroxide, aqueous ammonia, amines or alkanolamines;
the expression “at least one” is equivalent to “one or more”; and
the expression “inclusive” for a range of concentrations means that the limits of the range are included in the defined range.

For the purposes of the present invention, the term “photodimerizable group” means a chemical group that leads to photodimerization reactions under irradiation. For the purposes of the invention, photodimerization is a chemical reaction between two double bonds (of 2+2 type) or two pairs of double bonds (of 4+4 type), and more particularly between two double bonds (of 2+2 type).

The case of a reaction between two double bonds may be represented schematically in the following manner:

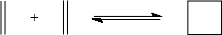

These photodimerization reactions are defined in the book Advanced Organic Chemistry, J. Marck, 4th edition, Wiley Interscience, NY 1992, page 855.

Thus, the double bond, when it is photo-stimulated, generally when it is subjected to UV radiation, proves to be capable of reacting with another double bond by cyclization.

According to the present invention, the double bond is said to be activated, i.e. it is spontaneously photodimerizable, without requiring the presence of a photoinitiator or a chemical initiator.

This double bond is generally activated by the presence of an electron-withdrawing substituent in the alpha position of this photodimerizable double bond. As electron-withdrawing substituent, mention may be made of aromatic nuclei such as the phenyl group optionally substituted with one or more halogen atoms, or electron-withdrawing groups such as NO₂, CN, R′—Y—C(Y′)—, R′—C(Y′)—Y—, R′—Y—C(Y′)—Y—, R′—Y—S(O)₂— or —S(O)₂—Y—R′, where R′ represents a hydrogen atom or a (C₁-C₄)alkyl group optionally substituted with one or more halogen atoms, where Y and Y′, which may be identical or different, represent an oxygen or sulfur atom or NR″ where R″ represents a hydrogen atom or a (C₁-C₆)alkyl group.

The composition (C) according to the invention is preferably a composition for dyeing keratin fibers such as the hair.

### Photocrosslinkable Polymer:

The composition (C) according to the invention comprises at least one photocrosslinkable polymer comprising at least one photodimerizable group and at least one hydrophobic group.

Preferably, the photodimerizable pendent groups that may be used according to the invention are chosen from monovalent radicals having the following formulae (I) and (II):

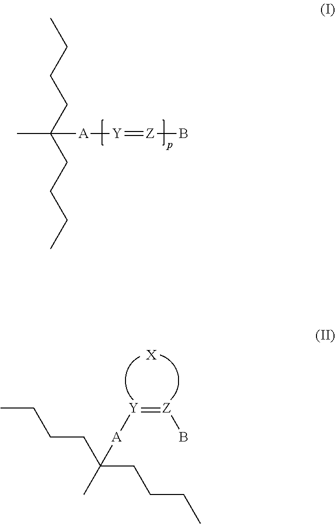

and also the geometrical isomers thereof,
formulae (I) and (II) wherein:
Y and Z independently denote a nitrogen atom or a group C(R) where R represents a hydrogen atom or a (C₁-C₄)alkyl group such as methyl;
A represents one bond or a divalent group chosen from (C₁-C₃)alkylene, arylene, heteroarylene, cycloalkylene, heterocycloalkylene, (thio)carbonyl, (C₂-C₃)alkenylene radicals and combinations thereof;
B represents a monovalent group chosen from (C₁-C₃)alkyl radicals, aryl, optionally cationic heteroaryl, cycloalkyl, optionally cationic heterocycloalkyl, (thio)carbonyl, (C₂-C₈)alkenyl radicals and combinations thereof;
X represents a divalent group chosen from (C₂-C₃)alkylene, arylene, heteroarylene, cycloalkylene, heterocycloalkylene, (thio)carbonyl, (C₂-C₈)alkenylene radicals and combinations thereof;
p represents an integer between 1 and 5 inclusive, more particularly between 1 and 3, preferably p is 1;

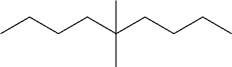

represents the bond that connects the part of the monovalent radical to the rest of the molecule;
and
where each of the groups cited can optionally be substituted with one or more halogen atoms or groups chosen from (C₁-C₆)alkyl, hydroxy, amino, (di)(C₁-C₆)alkylamino, phenyl, carboxy, (C₁-C₆)alkoxy, (C₁-C₆)alkoxy(thio)carbonyl, hydrogen(thio)-carbonyl, sulfonato R—O—S(O)₂— or R—S(O)₂—O—, amide RR′N—C(O)— or R—C(O)—N(R′)— or acyl R—C(O)—, ammonium RR′R″N⁺— groups, where R, R′, and R″, which may be identical or different, represent a hydrogen atom or a (C₁-C₄)alkyl group.

The pendent dimerizable groups according to the invention are especially those cited in U.S. Pat. No. 2,811,510, EP 0 313 220, EP 0 313 221, EP 092 901, GB 2 030 575 and GB 2 076 826, and in articles “Chemical Review Vol 83, 5 1983, p 507” “Polym, Paint color Journal 1988, 178, p 209” and “Current Trends in Polymer Photochemistry, Ellis Morwood edition, NY, 1995”.

As examples, photodimerizable pendent groups chosen from monovalent radicals from the following components may more particularly be cited:
stilbene,
styrylpyridinium (stilbazolium) having the formula below, and the geometric isomers thereof:

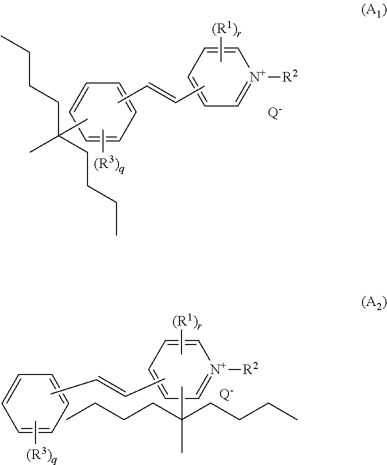

where:
R¹ and R³, which may be identical or different, represent a halogen atom or a (C₁-C₆)alkyl group; or alternatively two contiguous groups R¹ or R³ form, together with the carbon atoms that bear them, a benzo group;
R² represents a hydrogen atom, a (C₁-C₆)alkyl group optionally substituted with one or more halogen atoms such as chlorine, or hydroxyl; preferably, R² represents a (C₁-C₆)alkyl group such as methyl, ethyl or propyl;
q and r represent an integer between 0 and 4 inclusive; and
Q⁻ represents an anionic counterion preferably chosen from halide ions such as chlorides, bromides, iodides, perchlorates, tetrafluoroborates, methylsulfate, phosphates, sulfates, methanesulfonates, p-toluenesulfonate;

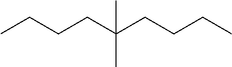

represents the bond that connects the part of the monovalent radical to the rest of the molecule, it being understood that the pendent group A₂ may be connected to the rest of the molecule via R²;

preferably the

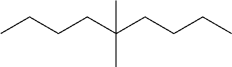

bond is found on the phenyl in the para position of the styryl group on A₁ or connected to the rest of the molecule via R² on A₂; preferentially the styryl group of A₁ and A₂ is found in the position para to the pyridinium group;
styrylazolium having the formula below, and the geometric isomers thereof:

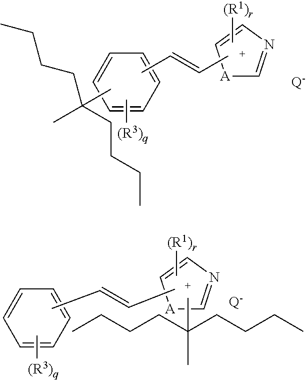

where:
A represents a sulfur atom, an oxygen atom, or a group NR² or C(R²)₂; and

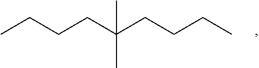

Q⁻, r, q, R¹, R², and R³ are as defined previously,
preferably the

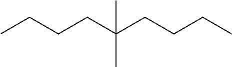

bond is found on the phenyl in the para position of the styryl group,
styrylpyrazine,
chalcone,
(thio)cinnamate and (thio)cinnamamide,
maleimide,
(thio)coumarin,
thymine,
uracil,
butadiene,
anthracene,
pyridone,
pyrrolizinone,
acridizinium salts,
furanone,
phenylbenzoxazole, and
derivatives thereof.

According to a particular embodiment, the photodimerizable pendent group(s) of the invention are chosen from:
a) photodimerizable group(s) bearing a stilbazolium function of formula (Ia) or (Ib) and the geometrical isomers thereof:

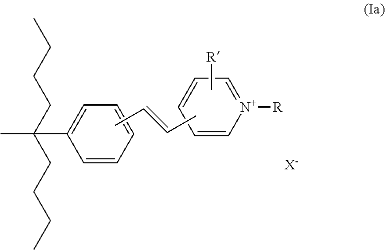

wherein:
R represents a hydrogen atom, or a C₁-C₄ alkyl or C₁-C₄ hydroxyalkyl group,
R′ represents a hydrogen atom or a C₁-C₄ alkyl group, and
X⁻ denotes an anionic counterion chosen from halide ions such as chlorides, bromides, iodides, perchlorates, tetrafluoroborates, methyl sulfate, phosphates, sulfate, methanesulfonate,

p-toluenesulfonate; preferably the styryl group is found in the position para to the pyridinium group and/or in the para-position to the

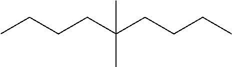

bond;

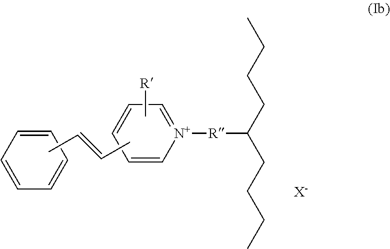

wherein
R″ denotes a divalent alkylene radical having from 2 to 8 carbon atoms,
R′ represents a hydrogen atom or a C₁-C₄ alkyl group, and
X⁻ has the same meaning as that described for the preceding formula (Ia);
where

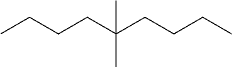

has the same meaning as previously;

preferably, the styryl group is located in the position para to the pyridinium group;

or
b) photodimerizable groups bearing a styrylazolium function of formula (IIa):

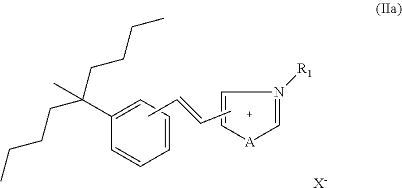

wherein:
R₁ denotes a hydrogen atom or a C₁-C₄ alkyl or C₁-C₄ hydroxyalkyl group;
A denotes a sulfur atom, an oxygen atom or an NR′ or C(R′)₂ group, R′; where R′ represents a hydrogen atom or a C₁-C₄ alkyl group, and R′ preferably represents a hydrogen atom; and
X⁻ has the same meaning as that described for the preceding formula (Ia);
where

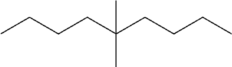

has the same meaning as previously;

preferably the styryl group is found on the phenyl group

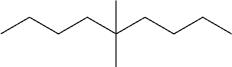

in the para position.

Such photodimerizable pendent groups bear activated double bonds, and as such the photodimerization of these double bonds starts spontaneously in the UVA range, without requiring a photoinitiator.

Photoinitiator is understood in the sense of the present invention as a compound that initiates the photodimerization reaction and releases a radical when irradiated, especially in the UV domain.

According to one particular embodiment, the composition of the invention is free of photoinitiators.

According to the present invention the polymer includes one or more pendent hydrophobic groups.

As pendent hydrophobic group, mention may be made of:
saturated or unsaturated (C₁-C₃₀)alkyl groups, optionally substituted and/or interrupted with one or more heteroatoms,
(C₂-C₃₀)alkenyl groups,
fluorinated groups such as fluorocarbon groups such as —CF₃, —CHF₂, —OCF₃, —SCF₃, CF₃C(O)—,
silicone groups such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, with Rₐ, R_{b} and R_{c}, which may be identical or different, representing a (C₁-C₃)alkyl group which is optionally interrupted and/or terminated with one or more non-contiguous heteroatoms such as O or S, and R representing a (C₁-C₆)alkyl group.

Preferably the pendent hydrophobic group(s) are chosen from a (C₂-C₂₂)alkyl or phenyl group, more preferentially from a (C₃-C₁₆)alkyl group, better still a (C₃-C₃)alkyl group.

The polymer backbone may have varied nature. This polymer backbone may be natural or synthetic. As natural polymer backbones, mention may be made of polysaccharides.

As polysaccharides, mention may be made of xanthan, carrageenan, chitosan, cellulose and its derivatives, alginate, starch, dextran, pullulan, galactomannan and the biologically acceptable salts thereof, and derivatives thereof.

As synthetic backbones, mention may be made of poly(vinyl) polymers and polydiorganosiloxanes.

Among the poly(vinyl) polymers, mention may be made of partially or totally hydrolyzed poly(vinyl acetate)s, and polyvinyl alcohol (PVA).

As regards the polymers containing photodimerizable pendent groups bearing a stilbazolium function, they are obtained by reacting the polymer under consideration with a chemical entity including a group having formula (Ia) or (Ib).

Preferably, the chemical species including a group (Ia) bears a reactive group W of aldehyde or acetal type.

As chemical entities that may be used to graft styrylpyridinium groups, mention may especially be made of quaternary salts of 2-(4-formylstyryl)pyridinium, 4-(4-formyl-styryl)pyridinium, 2-(3-formylstyryl)pyridinium, N-methyl-2-(4-formylstyryl)pyridinium, N-methyl-3-(4-formylstyryl)pyridinium, N-methyl-2-(3-formylstyryl)pyridinium, N-methyl-2-(2-formylstyryl)pyridinium, N-ethyl-2-(4-formylstyryl)pyridinium, N-(2-hydroxyethyl)-2-(4-formylstyryl)pyridinium, N-(2-hydroxyethyl)-4-(4-formylstyryl)pyridinium, N-methyl-4-(4-formylstyryl)pyridinium, N-methyl-4-(3-formylstyryl)pyridinium.

The pyridinium quaternary salts may be chloride, bromide, iodide, perchlorate, tetrafluoroborate, methosulfate, phosphate, sulfate, methanesulfonate or p-toluenesulfonate salts. Such chemical entities are described in GB-A-2030575.

Examples of species that may be mentioned include 4-(4-formylphenylethenyl)-1-methylpyridinium methosulfate, 1-(3-ethoxycarbonylmethyl)-4-[2-(4-formylphenyl)ethenyl]pyridinium bromide and 1-(methoxycarbonylpropyl)-4-[2-(4-formylphenyl)ethenyl]pyridinium bromide. Such species are described in US 2007/0 112 094.

Use is preferably made of n-methyl-4-(4-formylstyryl)pyridinium methyl sulfate (RN=74401-04-0), sold especially by Wako.

The synthesis, described below, of these polymers functionalized with photodimerizable groups such as those comprising a styryl group and hydrophobic groups may be performed on the basis of the protocol of T. Uhlich et al. (Reactive & Functional Polymers, 28, 55-40 (1995)).

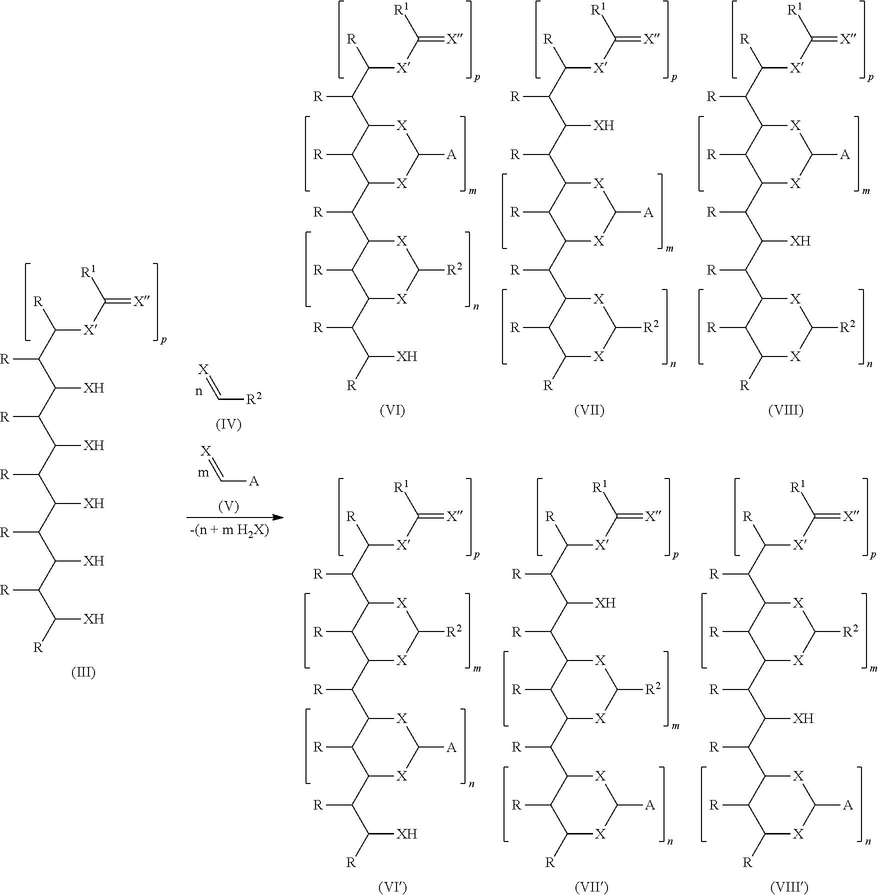

Compound having formula (III) to (VIII′) wherein,
R, identical or different, represents a hydrogen atom, or a (C₁-C₁₀)alkyl group, optionally substituted and/or interrupted by one or more heteroatoms, preferably R represents a hydrogen atom or a (C₁-C₄)alkyl group such as methyl, ethyl, or propyl, more preferentially R represents a hydrogen atom;
R¹ represents a hydrogen atom, or a (C₁-C₁₀)alkyl group, optionally substituted and/or interrupted by one or more heteroatoms, preferably R¹ represents a hydrogen atom or a (C₁-C₄)alkyl group;
R² represents a saturated or unsaturated (C₁-C₃₀)alkyl group, optionally substituted and/or interrupted by one or more heteroatoms, an alkenyl group, an aryl group such as phenyl, pyridyl, furyl, indoyl, benzofuryl, thiophenyl, imizadoyl, oxazoyl, thiazoyl, pyrazinyl, pyrimidinyl; a fluorinated group such as a fluorocarbon group such as —CF₃, —CHF₂, —OCF₃, —SCF₃, CF₃C(O)—, a silicone group such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, with Rₐ, R_{b} and R_{c}, which may be identical or different, representing a (C₁-C₃)alkyl group optionally interrupted and/or terminated by one or more non-contiguous heteroatoms such as O, or S; and where R represents a (C₁-C₆)alkyl group; preferably R² represents a (C₂-C₂₂)alkyl group, more preferentially (C₃-C₁₆)alkyl;
A represents a group from a photodimerizable compound, preferably styrylpyridinium, such as (1), (II), (A₁), (A₂), (Ia), (Ib) or (IIa) as defined previously, more particularly chosen from (A₁) or (Ia) as defined previously;
X represents an oxygen or sulfur, preferably oxygen, atom;
X′, and X″ represent an oxygen or sulfur atom or an N(R³) group with R³ denoting a hydrogen atom or a (C₁-C₄)alkyl group; preferably, X and X″ represent an oxygen atom;

Mainly the products obtained have formula (VI).

Advantageously, these chemical entities react with a polyvinyl alcohol or polyvinyl acetal type polymer as described in the documents cited previously and also such as polymer (III) described in the scheme above for which X, X′ and X″ represent an oxygen atom, R and R¹ being as described previously.

For example, a grafted polyvinyl alcohol polymer including the following structure units results, where A represents a group (I), (A₁) or (Ia):

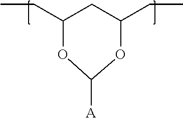

Polyvinyl alcohol polymers grafted with a styrylpyridinium group are especially described in the publication Ichimura K. et al., Preparation and characteristics of photocrosslinkable poly(vinyl alcohol), Journal of Polymer Science, Polymer Chemistry Edition, Vol. 20, 1419-1432 (1982).

The polymers may be obtained by reacting polyvinyl alcohol or partially hydrolyzed polyvinyl acetate with styrylpyridinium salts bearing a formyl or acetal group as described in GB-A-2 030 575, WO 96/29312, U.S. Pat. No. 5,061,603, GB-A-2 076 826 and EP-A-092 901.

Cellulose polymers grafted with styrylpyridinium groups are notably described in US 2007/0 112 094.

Preferably, the chemical entity including a group (A₁) or (Ia) bears one reactive group that is a halogen atom such as chlorine.

In this variant, the chemical entity meets for example the formula:

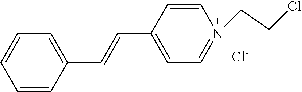

Advantageously, the photocrosslinkable compound comprising groups (Ib) is for example obtained by reacting the entity above with the polysaccharide chosen from those defined previously.

As regards the polymers containing photodimerizable groups bearing a styrylazolium function, they are obtained by reacting the polymer with a chemical entity comprising a group having formula (IIa).

Preferably, the chemical entity including a group (IIa) bears a reactive group W of aldehyde or acetal type.

As chemical species that may be used for grafting groups of styrylazolium type, mention may be made of those described in EP-A-313 220.

Advantageously, these chemical species react with a polymer of polyvinyl alcohol or polyvinyl acetate type as described in the documents mentioned previously.

A grafted polyvinyl alcohol polymer including the following structure units thus results with B corresponding to the group or (IIa) as defined previously:

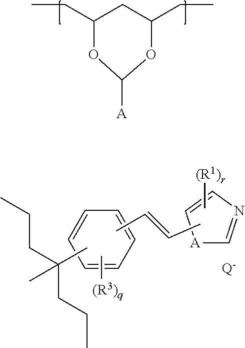

Polyvinyl alcohol polymers grafted with styrylazolium groups are notably described in EP-A-313 220. In said document, these polymers may be obtained by reaction of polyvinyl alcohol or partially hydrolyzed polyvinyl acetate with styrylazolium salts bearing an aldehyde or acetal group.

According to one embodiment, the polymer bearing (a) photodimerizable group(s) and (a) hydrophobic group(s) is in the form of particles, in particular of dispersed particles. Thus, in the latter case, the polymer particles are more particularly polyvinyl alcohol particles.

According to a preferred embodiment, the polymer bearing (a) photodimerizable group(s) and (a) hydrophobic group(s) of the invention is soluble in the cosmetic medium.

Thus, according to one embodiment variant, the polymer is a polyvinyl acetate (PVAc) polymer partly functionalized with one or more hydroxyl functions and one or more functions having formula (IX):

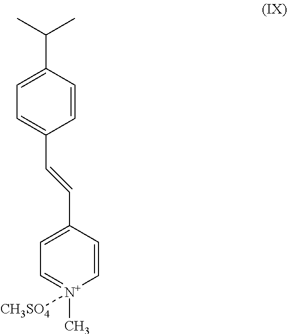

The degree of polymerization of the PVAc may be between 100 and 5000 and the degree of substitution, as a percentage of the functions of formula (I) as defined above, may be between 0.1% and 25%, preferably from 0.5% to 5%.

The following scheme represents one variant where the polymer is the polymer (Ill) as defined previously bearing functions grafted by stylbazolium entities such as those having formula (A1) as defined previously, which can crosslink under the effect of light, as illustrated below.

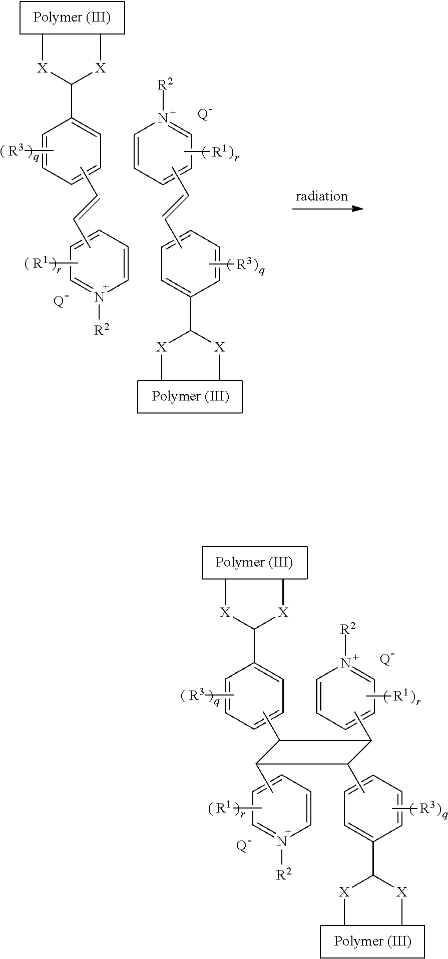

These materials react to radiation that may include both a UV light and visible light component, particularly a low dose of UV.

Preferentially, the following scheme represents the polymer which is PVA-SbQ (polymer of polyvinyl acetate type bearing a few hydrolyzed functions and a few functions grafted with stilbazolium entities), which can crosslink under the effect of light, as illustrated below.

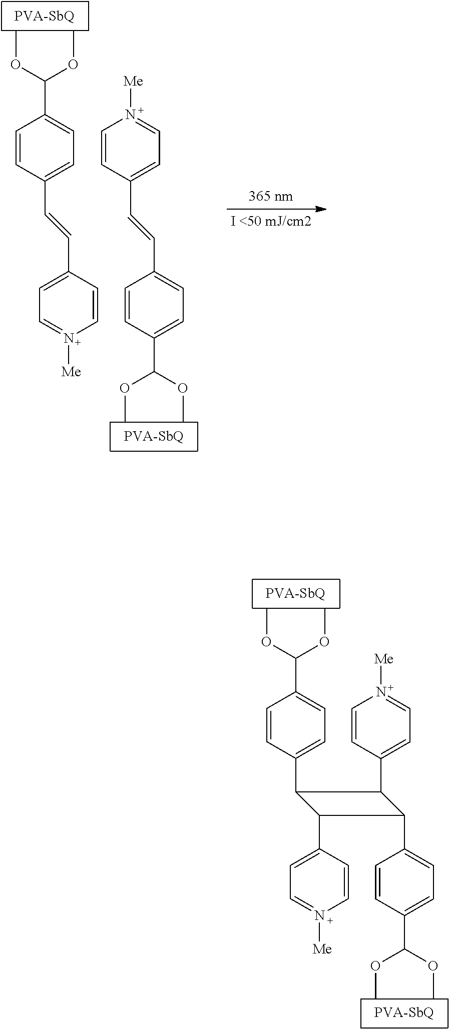

These materials are particularly appreciated since they do not require a photoinitiator and react with radiation that may include both UV light and visible light, in particular a low dose of UV.

Pendent groups which have reactivity both in UV light and in visible light are favored.

According to another embodiment variant, the photocrosslinkable compound is featured by a natural polymer that is functionalized with photodimerizable groups and hydrophobic groups.

It may notably be a polysaccharide that may notably be chosen from chondroitin sulfate, keratan, keratan sulfate, heparin, heparin sulfate, xanthan, carrageenan, hyaluronic acid, chitosan, cellulose and derivatives thereof, alginate, starch, dextran, pullulan, galactomannan and biologically acceptable salts thereof.

Needless to say, the degree of functionalization is adjusted be able to provide the degree of crosslinking required during activation.

According to the invention, the degree of functionalization with photodimerizable units is at least 0.1%, or even at least 0.5%, or even at least 2%.

Preferably, in a composition according to the invention, the photodimerizable groups are borne by a polyvinyl acetate, polyvinyl alcohol or polysaccharide polymer.

The crosslinkable polymer may be carried in an aqueous medium.

The composition may contain a single polymer bearing photodimerizable pendent groups that may or may not be of different nature.

Use may also be made of a mixture of polymers having different functions.

Consequently, the reactions may take place between two photodimerizable groups that may or may not be of the same chemical nature.

The activated double bonds may react with another double bond of the same chemical nature or may react with another double bond of different chemical nature.

As examples of polymers useful in the invention, mention may be made of the partially hydrolyzed polyvinyl acetate polymer comprising the units below, in particular the polymer called PVA-SbQ-propyl wherein the amount of SbQ units is inclusively between 0.5 and 6 mol %, preferably between 1.5 and 5 mol %, the amount of propyl groups is inclusively between 2 and 20 mol %, preferably between 5 and 15 mol %, better still between 8 and 12 mol %, the amount of hydroxyl groups is inclusively between 50 and 97.5 mol %, preferably between 60 and 97.5 mol %, better still from 80 to 90 mol %:

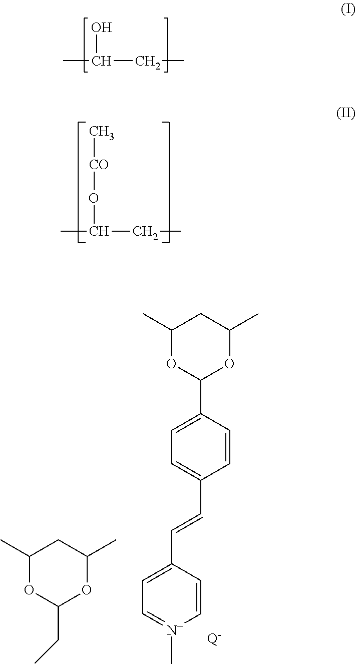

with Q⁻ representing an anionic counterion preferably chosen from halide ions such as chlorides, bromides, iodides, perchlorates, tetrafluoroborates, methylsulfate, phosphates, sulfates, methanesulfonates, p-toluenesulfonate, preferably mesylate CH₃OSO₃⁻;

According to one particular embodiment, the molecular weight Mw of the PVA is between 10 000 and 100 000 g/mol, preferably between 15 000 and 80 000 g/mol, more preferentially from 20 000 to 50 000, even better still from 25 000 to 35 000 g/mol.

The total amount of the polymer(s) comprising at least one photodimerizable group and at least one hydrophobic group, present in the composition according to the invention, preferably ranges from 0.01% to 40% by weight, more preferentially from 0.1% to 30% by weight, better still from 0.5% to 25% by weight, and even better still from 1% to 10% by weight, relative to the total weight of the composition C.

The composition according to the invention may also comprise an effective amount of at least one photosensitizing agent.

For the purposes of the present invention, the term “photosensitizer” means an ingredient which modifies the irradiation wavelength, thereby triggering the photodimerization reaction.

For example, the photodimerization of dimethylmaleimide groups is triggered by irradiation centered on the wavelength range from 270 to 300 nm. In the presence of a photosensitizer such as thioxanthone, photodimerization becomes effective with irradiation centered on the wavelength range from 360 to 430 nm.

Among the photosensitizers that may be used according to the invention, mention may notably be made of thioxanthone, rose Bengal, phloxine, eosin, erythrosine, fluorescein, acriflavine, thionine, riboflavin, proflavine, chlorophylls, haematoporphyrin, methylene blue and mixtures thereof.

In practice, the photosensitizing agent that may be used according to the invention represents 0.00001% to 5% of the total weight of the composition.

### Coloring Agent:

The composition (C) according to the invention comprises at least one coloring agent chosen from pigments and mixtures thereof.

Preferably, the composition (C) according to the invention comprises one or more pigments.

The term “pigment” refers to any pigment that gives color to keratin materials. Their solubility in water at 25° C. and at atmospheric pressure (760 mmHg) is less than 0.05% by weight, and preferably less than 0.01%.

The pigments that may be used are notably chosen from the organic and/or mineral pigments known in the art, notably those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” means any pigment that satisfies the definition in Ullmann's encyclopedia in the chapter on inorganic pigments.

Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopedia in the chapter on organic pigments.

The organic pigment may notably be chosen from nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or colored organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by Hoechst under the names:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may notably be composed of particles including a mineral core, at least one binder for attaching the organic pigments to the core, and at least one organic pigment which at least partially covers the core.

The organic pigment may also be a lake. The term “lake” refers to dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the dyes, mention may be made of carminic acid. Mention may also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). They thereby differ from colored pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica covered with titanium or with bismuth oxychloride, colored nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the Cellini nacres sold by BASF (mica-TiO2-lake), Prestige sold by Eckart (mica-TiO2), Prestige Bronze sold by Eckart (mica-Fe2O3), and Colorona sold by Merck (mica-TiO2-Fe2O3).

Mention may also be made of the gold-colored nacres sold notably by BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by Merck under the name Bronze fine (17384) (Colorona) and Bronze (17353) (Colorona) and by BASF under the name Super bronze (Cloisonne); the orange nacres sold notably by BASF under the name Orange 363C (Cloisonne) and Orange MCR 101 (Cosmica) and by Merck under the name Passion orange (Colorona) and Matte orange (17449) (Microna); the brown nacres sold notably by BASF under the name Nu-antique copper 340XB (Cloisonne) and Brown CL4509 (Chromalite); the nacres with a copper tint sold notably by BASF under the name Copper 340A (Timica); the nacres with a red tint sold notably by Merck under the name Sienna fine (17386) (Colorona); the nacres with a yellow tint sold notably by BASF under the name Yellow (4502) (Chromalite); the red nacres with a gold tint sold notably by BASF under the name Sunstone G012 (Gemtone); the pink nacres sold notably by BASF under the name Tan opale G005 (Gemtone); the black nacres with a gold tint sold notably by BASF under the name Nu antique bronze 240 AB (Timica), the blue nacres sold notably by Merck under the name Matte blue (17433) (Microna), the white nacres with a silvery tint sold notably by Merck under the name Xirona Silver, and the golden-green pink-orange nacres sold notably by Merck under the name Indian summer (Xirona), and mixtures thereof.

Still as examples of nacreous agents, mention may also be made of particles including a borosilicate substrate coated with titanium oxide.

Particles comprising a glass substrate coated with titanium oxide are notably sold under the name Metashine MC1080RY by Toyal.

Finally, examples of nacres that may also be mentioned include polyethylene terephthalate glitter flakes, notably those sold by Meadowbrook Inventions under the name Silver 1P 0.004×0.004 (silver glitter flakes). It is also possible to envisage multilayer pigments based on synthetic substrates, such as alumina, silica, calcium sodium borosilicate, calcium aluminium borosilicate and aluminium.

The pigments with special effects may also be chosen from reflective particles, i.e. notably from particles of which the size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. brighter points that contrast with their environment, making them appear to sparkle.

The reflective particles may be selected so as not to significantly alter the coloring effect generated by the coloring agents with which they are combined, and more particularly so as to optimize this effect in terms of color rendition. They may more particularly have a yellow, pink, red, bronze, orange, brown, gold and/or coppery color or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminum powder, bronze powder or copper powder coated with SiO₂ sold under the name Visionaire by Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by Quantum Dots Corporation.

The variety of pigments that may be used in the present invention makes it possible to obtain a wide range of colors, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C₈ to C₂₀ fatty acid and of polyols such as glycerol or diglycerol are used, such as poly(12-hydroxystearic acid) stearate with a molecular weight of approximately 750 g/mol, such as the product sold under the name Solsperse 21 000 by Avecia, polyglyceryl-2 dipolyhydroxystearate (CTFA name) sold under the reference Dehymyls PGPH by Henkel, or polyhydroxystearic acid such as the product sold under the reference Arlacel P100 by Uniqema, and mixtures thereof.

As other dispersants that may be used in the compositions of the invention, mention may be made of quaternary ammonium derivatives of polycondensed fatty acids, for instance Solsperse 17 000 sold by Avecia, and polydimethylsiloxane/oxypropylene mixtures such as those sold by Dow Corning under the references DC2-5185 and DC2-5225 C.

The pigments used in the composition may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminum salts of fatty acids, for example aluminum stearate or laurate; metal alkoxides; polyethylene; (meth)acrylic polymers, for example polymethyl methacrylates; polymers and copolymers containing acrylate units; alkanolamines; silicone compounds, for example silicones, in particular polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in U.S. Pat. No. 4,578,266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminium dimyristate treatment, for instance the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or inorganic pigments in submicron-sized particulate form in the dye composition.

The term “submicron-sized” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in a (dispersant:pigment) amount of between 1:4 and 4:1, particularly between 1.5:3.5 and 3.5:1 or better still between 1.75:3 and 3:1.

The dispersant(s) may thus have a silicone backbone, such as silicone polyether and dispersants of amino silicone type other than the alkoxysilanes described previously.

Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type other than the silicones described previously and are cationic.

Preferably, the pigment(s) are chosen from mineral, mixed mineral-organic or organic pigments.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

The pigment(s) may be present in a total content ranging from 0.001% to 20% by weight, preferably from 0.1% to 15% by weight and better still from 1% to 10% by weight, relative to the total weight of the composition (C).

The composition (C) may also comprise one or more direct dye(s).

The term “direct dye” is understood to mean natural and/or synthetic dyes, other than oxidation dyes. These are dyes which will spread superficially over the fiber.

They may be ionic or nonionic, preferably cationic or nonionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (XII) and (XIII) and the azo cationic dyes (XIV) and (XV) below:

[Chem. 29]

Hét⁺—C(R^{a})═N—N(Rb)—Ar,Q (XII)

[Chem. 30]

Hét⁺—N(Ra)—N═C(Rb)—Ar,Q- (XIII)

[Chem. 31]

Hét⁺—N═N—Ar,Q- (XIV)

[Chem. 32]

Ar⁺—N═N—Ar″,Q- (XV)

formulae (XII) to (XV) wherein:
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group;
   or else the substituent R^{a} with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (XII) to (XV) as defined previously, more particularly the cationic direct dyes bearing an endocyclic cationic charge described in patent applications WO 95/15144, WO 95/01772 and EP 714 954, preferentially the following direct dyes:

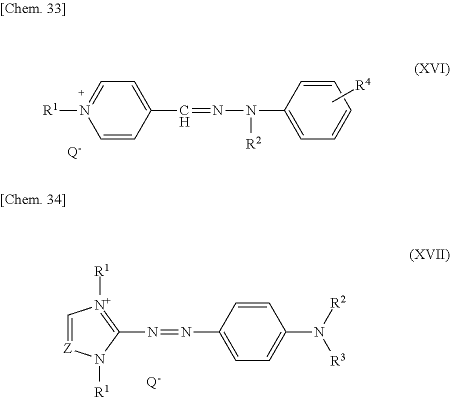

formulae (XVI) and (XVII) wherein:
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₃)alkyl, optionally substituted (C₁-C₃)alkoxy, or (di)(C₁-C₃)(alkyl)amino optionally substituted on the alkyl group(s) with a hydroxyl group; particularly, R⁴ is a hydrogen atom,
Z represents a CH group or a nitrogen atom, preferentially CH,
Q- is an anionic counterion as defined previously, in particular a halide, such as chloride, or an alkyl sulfate, such as methyl sulfate or mesyl.

In particular, the dyes of formulae (XVI) and (XVII) are chosen from Basic Red 51, Basic Yellow 87 and Basic Orange 31 or derivatives thereof with Q- being an anionic counterion as defined previously, particularly halide such as chloride, or an alkyl sulfate such as methyl sulfate or mesityl.

The direct dyes may be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

By way of acid dyes according to the invention, mention may be made of the dyes of formulae (XVIII), (XVIII′), (XIX), (XIX′), (XX), (XX′), (XXI), (XXI′), (XXII), (XXIII), (XXIV) and (XXV) below:
a) the diaryl anionic azo dyes of formula (XIII) or (XVIII′):

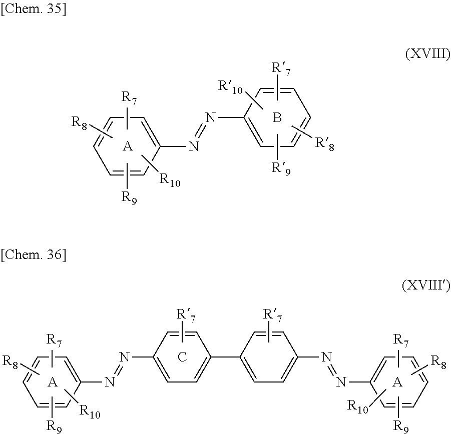

formulae (XVIII) and (XVIII′) wherein:
R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
R″—S(O)₂—, with R″ representing a hydrogen atom or an alkyl, aryl, (di)(alkyl)amino or aryl(alkyl)amino group; preferentially a phenylamino or phenyl group;
R′″—S(O)₂—X′— with R′″ representing an optionally substituted alkyl or aryl group, X′ as defined previously;
(di)(alkyl)amino;
aryl(alkyl)amino optionally substituted with one or more groups chosen from i) nitro; ii) nitroso; iii) (O)₂S(O⁻)—, M⁺ and iv) alkoxy with M⁺ as defined above;
optionally substituted heteroaryl; preferentially a benzothiazolyl group;
cycloalkyl; notably cyclohexyl;
Ar—N═N— with Ar representing an optionally substituted aryl group, preferentially a phenyl optionally substituted with one or more alkyl, (O)₂S(O⁻)—, M⁺ or phenylamino groups;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)—; ix) R^{∘}—X′—C(X)—X″—; x) Ar—N═N— and xi) optionally substituted aryl(alkyl)amino; with M⁺, R^{∘}, X, X′, X″ and Ar as defined previously;
W represents a sigma bond □, an oxygen or sulfur atom, or a divalent radical i) —NR— with R as defined previously, or ii) methylene —C(Ra)(Rb)- with Ra and Rb, which may be identical or different, representing a hydrogen atom or an aryl group, or alternatively Ra and Rb together form, with the carbon atom that bears them, a spiro cycloalkyl; preferentially, W represents a sulfur atom or Ra and Rb together form a cyclohexyl;
it being understood that formulae (XVIII) and (XVIII′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

As examples of dyes of formula (XVIII), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment Red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;
and by way of examples of dyes of formula (XVIII′), mention may be made of: Acid Red 111, Acid Red 134, Acid Yellow 38;
b) pyrazolone anionic azo dyes of formulae (XIX) and (XIX′):

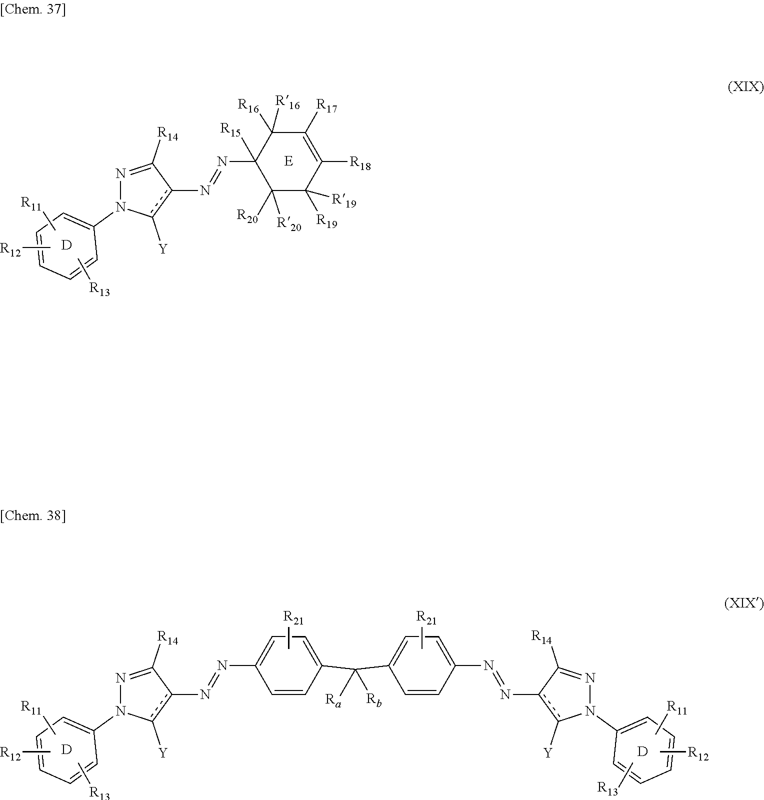

formulae (XIX) and (XIX′) wherein:
R₁₁, R₁₂ and R₁₃, which may be identical or different, represent a hydrogen or halogen atom, an alkyl group or —(O)₂S(O—), M⁺ with M⁺ as defined previously;
R₁₄ represents a hydrogen atom, an alkyl group or a group —C(O)O⁻, M⁺ with M⁺ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, or a group chosen from:
(O)₂S(O⁻)—, M⁺ with M⁺ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Rₐ and R_{b}, which may be identical or different, are as defined previously, preferentially Rₐ represents a hydrogen atom and R_{b} represents an aryl group;
Y represents either a hydroxyl group or an oxo group;
[symbol] represents a single bond when Y is an oxo group; and represents a double bond when Y represents a hydroxyl group;
   it being understood that formulae (XIX) and (XIX′) comprise at least one sulfonate radical (O)₂S(O⁻)—, M⁺ or one carboxylate radical —C(O)O⁻, M⁺ on one of the rings D or E;
   preferentially sodium sulfonate;

As examples of dyes of formula (XIX), mention may be made of: Acid Red 195, Acid Yellow 23, Acid Yellow 27 or Acid Yellow 76, and mention may be made, as examples of dyes of formula (XIX′), of: Acid Yellow 17;
c) the anthraquinone dyes of formulae (XX) and (XX′):

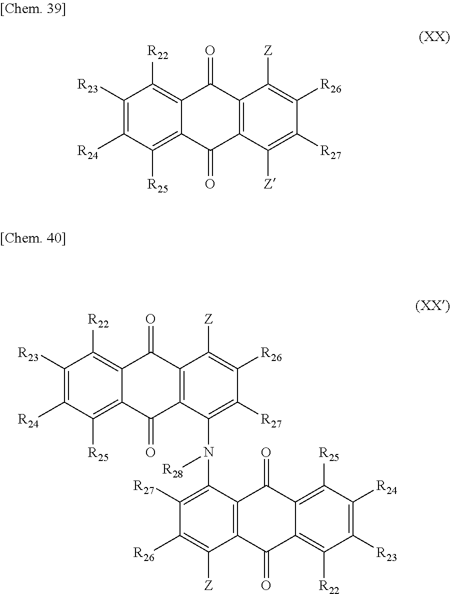

formulae (XX) and (XX′) wherein:
R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or arylthio, preferentially substituted with one or more groups chosen from alkyl and (O)₂S(O⁻)—, M⁺ with M⁺ as defined previously;
aryl(alkyl)amino optionally substituted with one or more groups chosen from alkyl and (O)₂S(O⁻)—, M⁺ with M⁺ as defined above;
(di)(alkyl)amino;
(di)(hydroxyalkyl)amino;
(O)₂S(O⁻)—, M⁺ with M⁺ as defined previously;
   Z′ represents a hydrogen atom or a group NR28R29 with R28 and R29, which may be identical or different, representing a hydrogen atom or a group chosen from:
alkyl;
polyhydroxyalkyl such as hydroxyethyl;
aryl optionally substituted with one or more groups, particularly i) alkyl such as methyl, n-dodecyl, n-butyl; ii) (O)₂S(O⁻)—, M⁺ with M⁺ as defined previously; iii) R^{∘}—C(X)—X′—, R^{◯}—X′—C(X)—, R^{∘}—X′—C(X)—X″— with R^{∘}, X, X′ and X″ as defined previously, preferentially R^{∘} represents an alkyl group;
cycloalkyl; notably cyclohexyl;
   Z, represents a group chosen from hydroxyl and NR′28R′29 with R′28 and R′29, which may be identical or different, representing the same atoms or groups as R28 and R29 as defined previously;
   it being understood that formulae (XX) and (XX′) comprise at least one sulfonate radical (O)₂S(O⁻)—, M⁺ or one carboxylate radical C(O)O⁻, M⁺; preferentially sodium sulfonate.

As examples of dyes of formula (XX), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet No. 2; and, as an example of a dye of formula (XX′), mention may be made of: Acid Black 48.
d) the nitro dyes of formulae (XXI) and (XXI′):

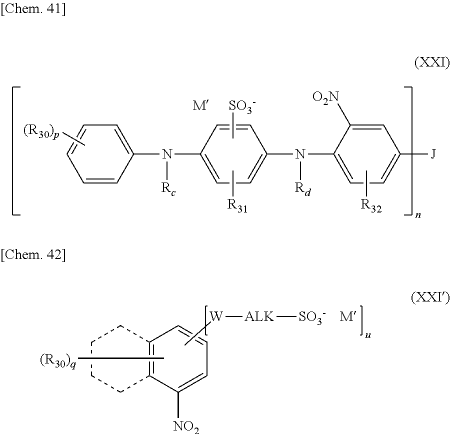

formulae (XXI) and (XXI′) wherein:
R₃₀, R₃₁ and R₃₂, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R^{∘}—C(X)—X′—, R^{∘}—X′—C(X)—, R^{∘}—X′—C(X)—X″— with R^{∘}, X, X′ and X″ as defined previously;
(O)₂S(O⁻)—, M⁺ with M⁺ as defined previously;
(O)CO⁻—, M⁺ with M⁺ as defined previously;
(di)(alkyl)amino;
(di)(hydroxyalkyl)amino;
heterocycloalkyl such as piperidino, piperazino or morpholino; in particular, R₃₀, R₃₁ and R₃₂ represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a —CH₂—CH₂— group;
n is 1 or 2;
p represents an integer inclusively between 1 and 5;
q represents an integer inclusively between 1 and 4;
u is 0 or 1;
when n is 1, J represents a nitro or nitroso group; particularly nitro;
when n is 2, J represents an oxygen or sulfur atom, or a divalent radical —S(O)m- with m representing an integer 1 or 2; preferentially, J represents an —SO₂— radical;
M′ represents a hydrogen atom or a cationic counterion;

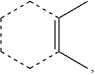

which may be present or absent, represents a benzo group optionally substituted with one or more groups R30 as defined above;
   it being understood that formulae (XXI) and (XXI′) comprise at least one sulfonate radical (O)₂S(O⁻)—, M⁺ or one carboxylate radical C(O)O⁻, M⁺; preferentially sodium sulfonate.

As examples of dyes of formula (XXI), mention may be made of: Acid Brown 13 and Acid Orange 3; as examples of dyes of formula (XXI′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; EXT D&C Yellow 7;
d) the triarylmethane dyes of formula (XXII):

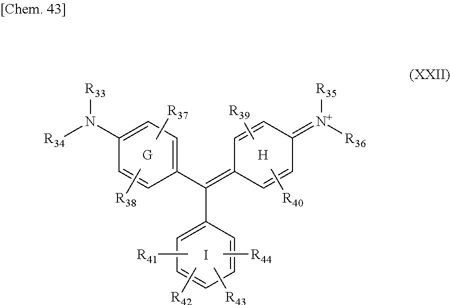

formula (XXII) wherein:
R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined above;
R₃₇, R₃₈, R₃₉, R₄₀, R₄₁, R₄₂, R₄₃ and R₄₄, which may be identical or different, represent a hydrogen atom or a group chosen from:
alkyl;
alkoxy, alkylthio;
(di)(alkyl)amino;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)— and ix) R^{∘}—X′—C(X)—X″—; with M⁺, R^{∘}, X, X′ and X″ as defined previously;
   in particular, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with an (O)₂S(O⁻)— group;
   it being understood that at least one of the rings G, H, I or I′ comprises at least one sulfonate radical (O)₂S(O⁻)— or one carboxylate radical —C(O)O—; preferentially sulfonate.

As examples of dyes of formula (XXII), mention may be made of: Acid Blue 1, Acid Blue 3, Acid Blue 7, Acid Blue 9, Acid Violet 49, Acid Green 3, Acid Green 5 and Acid Green 50;
e) the xanthene-based dyes of formula (XXIII):

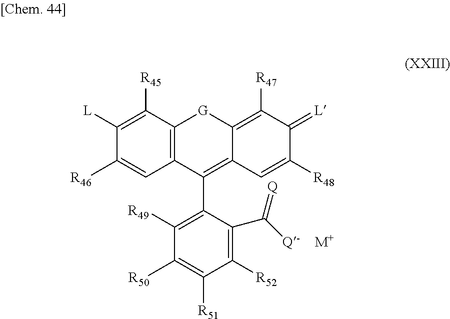

formula (XXIII) wherein:
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined previously;
particularly, R₅₃, R₅₄, R₅₅ and R₄₈ represent a hydrogen or halogen atom;
G represents an oxygen or sulfur atom or a group NRe with Re as defined previously; particularly, G represents an oxygen atom;
L represents an alkoxide O⁻, M⁺; a thioalkoxide S⁻, M⁺ or a group NRf, with Rf representing a hydrogen atom or an alkyl group, and M⁺ as defined previously; M⁺ is particularly sodium or potassium;
L′ represents an oxygen or sulfur atom or an ammonium group: N⁺RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly, Q and Q′ represent an oxygen atom;
M⁺ is as defined previously.

As examples of dyes of formula (XXIII), mention may be made of: Acid Yellow 73; Acid Red 51; Acid Red 52; Acid Red 87; Acid Red 92; Acid Red 95; Acid Violet 9;
f) the indole-based dyes of formula (XXIV):

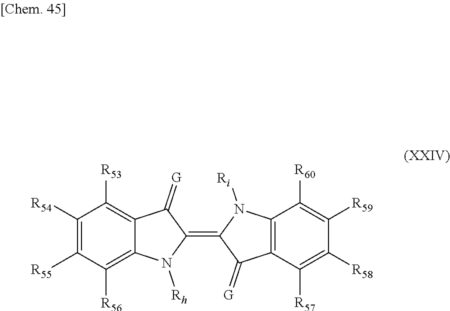

R₅₃, R₅₄, R₅₅, R₅₆, R₅₇, R₅₈, R₅₉ and R₆₀, which may be identical or different, represent a hydrogen atom or a group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined previously;
G represents an oxygen or sulfur atom or a group NRe with Re as defined previously; particularly, G represents an oxygen atom;
Ri and Rh, which may be identical or different, represent a hydrogen atom or an alkyl group;
it being understood that formula (XXIV) comprises at least one sulfonate radical (O)₂S(O⁻)—, M⁺ or one carboxylate radical —C(O)O⁻, M⁺; preferentially sodium sulfonate.

As an example of dyes of formula (XXIV), mention may be made of: Acid Blue 74.
g) the quinoline-derived dyes of formula (XXV):

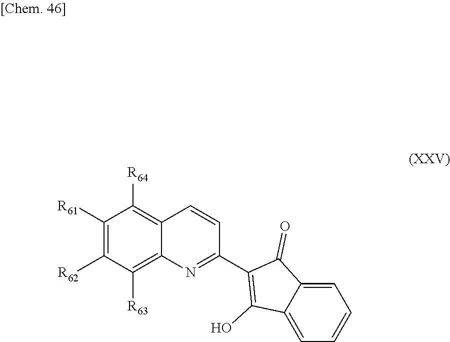

R₆₁ represents a hydrogen or halogen atom or an alkyl group;
R₆₂, R₆₃ and R₆₄, which may be identical or different, represent a hydrogen atom or a group (O)₂S(O⁻)—, M⁺ with M⁺ representing a hydrogen atom or a cationic counterion;
or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
it being understood that formula (XXV) comprises at least one sulfonate radical (O)₂S(O⁻)—, M⁺ preferentially sodium sulfonate.

As an example of dyes of formula (XXV), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dyes are chosen from anionic direct dyes.

The direct dye(s) may be present in a total content ranging from 0.001% to 10% by weight relative to the total weight of the composition, preferably from 0.005% to 5% by weight relative to the total weight of composition (C).

The coloring agent(s) may be present in a total content ranging from 0.001% to 20% by weight and preferably from 0.005% to 15% by weight relative to the total weight of composition (C).

Composition (C) according to the invention may comprise water. Preferably, water is present in a content ranging from 0.1% to 95% by weight, more preferentially from 1% to 92% by weight and better still from 10% to 90% by weight relative to the total weight of the composition.

### Organic Solvents:

Composition (C) according to the invention may comprise one or more organic solvents.

Examples of organic solvents that may be mentioned include lower C₁-C₄ alkanols, such as ethanol and isopropanol; polyols and polyol ethers, for instance glycerol, 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

The organic solvent(s) may be present in a total amount inclusively between 0.1% and 20% by weight approximately relative to the total weight of the dye composition, preferably between 0.5% and 15% by weight and more preferentially inclusively between 1% and 15% by weight relative to the total weight of composition (C).

### Additives:

Composition (C) may also contain any adjuvant or additive usually used.

Mention may be made, among the additives capable of being contained in the composition, of reducing agents, thickeners, softeners, antifoams, moisturizers, UV-screening agents, peptizers, solubilizers, fragrances, anionic, cationic, nonionic or amphoteric surfactants, proteins, vitamins, fixing polymers, conditioning agents, in particular polymers, preserving agents, waxes and mixtures thereof.

The composition according to the invention can be provided in particular in the form of a suspension, a dispersion, a gel, an emulsion, in particular an oil-in-water (O/W) or water-in-oil (W/O) emulsion, or a multiple (W/O/W or polyol/O/W or O/W/O) emulsion, in the form of a cream, a foam, a stick, a dispersion of vesicles, in particular of ionic or nonionic lipids, or a two-phase or multiphase lotion.

A person skilled in the art may select the appropriate presentation form, and also the method for preparing it, on the basis of his general knowledge, taking into account firstly the nature of the constituents used, notably their solubility in the support, and secondly the intended application of the composition.

### Protocol:

The present invention also relates to a process for the cosmetic treatment, in particular the dyeing, of keratin fibers such as the hair, wherein the composition (C) as defined above is applied to said fibers.

The composition (C) described above may be used on wet or dry keratin fibers, and also on any type of fair or dark, natural or dyed, permanent-waved, bleached or relaxed fibers.

According to one particular embodiment of the invention, the keratin fibers are washed before applying the composition (C).

The application to the fibers may be performed via any conventional means, in particular using a comb, a fine brush, a coarse brush, a sponge or with the fingers.

The application of the composition (C) to the keratin fibers is generally performed at ambient temperature (between 15 and 25° C.).

Preferably, after applying the composition (C) to the keratin fibers, there is a wait for between 1 minute and 6 hours, in particular between 1 minute and 2 hours, more particularly between 1 minute and 1 hour, more preferentially between 1 minute and 30 minutes, before, for example, a washing, rinsing, draining or drying step.

Preferably, a washing, rinsing, draining or drying step is performed after applying the composition (C) to the keratin fibers.

After application of the composition (C), the fibers may be left to dry or may be dried, for example at a temperature of greater than or equal to 30° C.

The process of the invention comprises, after step a) of applying the composition, optionally a drying step, and then a step b) of irradiating the composition on the keratin substances to crosslink the polymer.

This irradiation may be constituted of illumination, with ambient light or with a source of artificial light, of the composition applied to the keratin substances.

The ambient or artificial light may emit radiation in the visible and/or UV range.

Preferably, it emits at least a proportion of radiation in the UV range, for example a UV proportion of at least 2% of the total illuminating energy of the ambient light.

According to a particular embodiment, the exposure comprises, or even is constituted of, illumination with ambient light of the surface of said layer, in particular for a time of at least 1 minute.

The time of exposure to the ambient light may range more particularly from 10 seconds to 30 minutes and notably from 2 to 15 minutes.

According to another particular embodiment, the exposure comprises, or even is constituted of, illumination with a source of artificial light of the surface of said layer.

The time of exposure to said artificial light may range from 1 second to 20 minutes and in particular from 1 second to 1 minute.

The crosslinking may take place with natural or artificial light, for example using lighting with a lamp, a flash, a laser or LEDs, for example in the form of an LED array.

The artificial light source may emit radiation in the visible range and/or radiation in the UV range.

The light emitted may or may not be monochromatic. The wavelength of the emitted light is preferably centered on 365 nm, in particular between 100 nm and 500 nm and better still between 200 nm and 420 nm.

Advantageously, the crosslinking is initiated by simple illumination without the need for a photoinitiator.

Preferably, it will be a source of artificial light emitting energy between 0.5 and 5 W/cm², the exposure times being adapted in consequence.

The crosslinking may occur with reduced light intensity; the lighting system may produce this light intensity for example between 500 mJ/cm² and 10 J/cm².

The twofold characteristic of the absence of a photoinitiator and the relatively low light intensity is particularly advantageous since it makes it possible to limit the harmful effects of aggressive initiators or of prolonged exposure to intense light, in particular in the UV wavelengths.

A person skilled in the art will be capable of adapting the illumination characteristics, especially in terms of exposure time and of radiation wavelengths, with regard to the nature of the photocrosslinkable polymer(s) (A) used.

According to a preferred embodiment, the composition is applied to keratin fibers such as the hair.

According to this embodiment, the composition may be applied to wet or dry, clean or unclean keratin fibers, preferably wet and preferably clean keratin fibers. Preferably, the keratin fibers are dried after applying the composition and before irradiation.

In the process, before or after irradiation step b), a pause at ambient temperature, or high temperature, or under red light, may be included in the process.

Preferably, the step of applying composition (C) to the keratin fibers is repeated several times.

The present invention also relates to the use of composition (C) as defined above, for the cosmetic treatment of, in particular for dyeing, keratin fibers such as the hair.

The present invention will now be described more specifically by means of examples, which do not in any way limit the scope of the invention. However, the examples make it possible to support specific characteristics, variants and preferred embodiments of the invention.

### EXAMPLE

### Example 1

The following dye composition C1 is prepared:

Composition: amounts expressed in g of starting material as obtained/100 g

TABLE 1
C1

| | |
|---|---|
| Propyl-PVA-SbQ (Polyquaternium-114 PPVA-SBQ | 40.3 g |
| from MURAMI) in an aqueous solution containing | (6% am) |
| 15% of active material | |
| Mica and iron oxide (Pearlescent Pigment | 6.5 g |
| Prestige Soft Bronze from Sudarshan Chemical) | |
| Mixture of amomethicone dimethicone in an | 15 g |
| aqueous emulsion (Belsil DADM 5050^{E} from | (7.5% AM) |
| Wacker) | |
| Water qs | 100 g |

### Protocol:

Composition C1 is applied to locks of dry natural hair containing 90% white hairs, at a rate of 0.5 g of composition per gram of lock.

The locks of hair are then dried with a hairdryer with a round or flat brush or a comb, and then combed.

The hair treated was irradiated under UVA at 365 nm for an energy of 8 J/cm².

The locks of hair are left at ambient temperature for 24 hours.

Next, the locks of hair thus dyed are subsequently subjected to a test of several repeated shampoo washes so as to evaluate the fastness (persistence) of the obtained coloring with respect to shampoo washing.

### Shampoo Washing Protocol:

The locks of dyed hair are combed, moistened with water at 35° C. and then passed between the fingers five times for 5 seconds. The locks of hair are then squeezed dry between two fingers.

A standard shampoo (Garnier Ultra Doux) is applied uniformly to the colored locks, in a proportion of 0.4 g of standard shampoo per gram of locks, the locks of hair being massaged gently along the length (6 passes) for 15 seconds, from the root to the end.

The locks of hair are then placed on a watch glass and left to stand for 1 minute.

Next, the locks of hair are rinsed with water while passing the lock between the fingers (15 passes). The locks of hair are then squeezed dry between two fingers before the next shampoo wash.

Once the tests of several shampoo washes have been performed, the locks of hair are combed and dried with a hairdryer.

### Results:

The persistence of the color of the locks was evaluated in the CIE L*a*b* system, using a Minolta Spectrophotometer CM3600A colorimeter (illuminant D65, angle 10°, specular component included).

In this L*a*b* system, L* represents the intensity of the color, a* indicates the green/red color axis and b* the blue/yellow color axis.

The persistence of the coloring is evaluated by the color difference ΔE between the colored locks before shampooing, then after having undergone five shampoo washes according to the protocol described above. The lower the ΔE value, the more persistent the color with respect to shampoo washing.

The ΔE value is calculated according to the following equation:

$$Δ E = \sqrt{{( {L}^{⋆} - {L}_{o}^{⋆} )}^{2} + {( {a}^{⋆} - {a}_{o}^{⋆} )}^{2} + {( {b}^{⋆} - {b}_{o}^{⋆} )}^{2}} \qquad [ Math . 1 ]$$

In this equation, L*a*b* represent the values measured after coloring the hair and after performing the shampoo washes, and L₀*a₀*b₀* represent the values measured after coloring the hair but before shampoo washing.

TABLE 2

| | Number of | | | | |
|---|---|---|---|---|---|
| | shampoo | | | | |
| | washes | L* | a* | b* | ΔE |
| Composition | 0 | 36.0 | 29.0 | 22.3 | 0 |
| C1 | 5 | 37.4 | 27.4 | 20.9 | 2.6 |

The difference in ΔE is small. The locks of hair dyed with the composition C and washed with five shampoo washes show good persistence of the color with respect to shampoo washing.

### Example 3

The composition below was prepared:

amounts expressed in g of starting material as obtained/100 g

TABLE 3
C2

| | |
|---|---|
| Propyl-PVA-SbQ | 40.3 g (6% am) |
| Mica and iron oxide (Pearlescent Pigment | 4.23 |
| Prestige Soft Bronze from Sudarshan Chemical) | |
| Black iron oxide | 0.33 |
| Yellow iron oxide | 1.95 |
| Titanium oxide-coated glass particles | 1.5 |
| Mixture of amomethicone dimethicone in an | 15 g |
| aqueous emulsion (Belsil DADM 5050^{E} from | (7.5% AM) |
| Wacker) | |
| Water qs | 100 g |

Composition C2 was applied to locks of wet natural hair containing 90% white hairs, at a rate of 0.5 g of composition per gram of lock.

The locks of hair were then dried with a hairdryer, and then combed.

The hair treated was irradiated under UVA at 365 nm for an energy of 8 J/cm².

The locks of hair are left at ambient temperature for 24 hours.

### Results:

The locks of hair treated with composition C′ show smooth, uniform, chocolate colored coating on the hair with good cosmetic properties, notably in terms of softness, feel and good hair strand separation. The colored coating of the keratin fibers shows good persistence with respect to shampoo washing.

1. A composition (C) for the treatment of keratin fibers, comprising:
a. at least one photocrosslinkable polymer comprising at least one photodimerizable group and at least one hydrophobic group, and
b. at least one coloring agent chosen from pigments and mixtures thereof,
the hydrophobic group(s) being chosen from:
a saturated or unsaturated (C₁-C_{3W})alkyl group, optionally substituted and/or interrupted with one or more heteroatoms,
an alkenyl group,
a fluorinated group such as a fluorocarbon group such as —CF₃, —CHF₂, —OCF₃, —SCF₃, CF₃C(O)—,
a silicone group such as —SiRₐR_{b}R_{c} such as —Si(CH₃)₃, polydimethylsiloxane-PDMS, —Si(OR)₃, α,ω-diaminopropyl PDMS, α,ω-dihydroxyalkyl PDMS, α,ω-dicarboxyalkyl PDMS, with Rₐ, R_{b} and R_{c}, which may be identical or different, representing a (C₁-C₈)alkyl group which is optionally interrupted and/or terminated with one or more non-contiguous heteroatoms such as O or S, and R representing a (C₁-C₆)alkyl group.

2. The composition as claimed in claim 1, characterized in that the photodimerizable group(s) is (are) chosen from monovalent radicals having the following formulae (I) and (II): 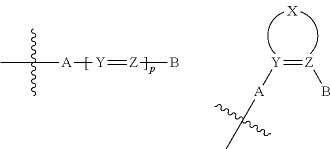
and also the geometrical isomers thereof,
formulae (I) and (II) wherein:
Y and Z denote, independently of each other, a nitrogen atom or a C(R) group with R representing a hydrogen atom or a (C₁-C₄)alkyl group such as methyl;
A represents a bond or a divalent group chosen from (C₁-C₈)alkylene, arylene, heteroarylene, cycloalkylene, heterocycloalkylene, (thio)carbonyl and (C₂-C₈)alkenylene radicals and combinations thereof;
B represents a monovalent group chosen from (C₁-C₈)alkyl radicals, aryl radicals, optionally cationic heteroaryl radicals, cycloalkyl radicals, optionally cationic heterocycloalkyl radicals, (thio)carbonyl radicals and (C₂-C₈)alkenyl radicals and combinations thereof;
X represents a divalent group chosen from (C₂-C₈)alkylene, arylene, heteroarylene, cycloalkylene, heterocycloalkylene, (thio)carbonyl and (C₂-C₈)alkenylene radicals and combinations thereof;
p represents an integer between 1 and 5 inclusive, in particular between 1 and 3;
preferably, p is equal to 1; 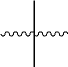
represents the bond that connects the part of the monovalent radical to the rest of the molecule;
and
it being possible for each of the cited groups to be optionally substituted with one or more halogen atoms or group(s) chosen from (C₁-C₆)alkyl, hydroxyl, amino, (di)(C₁-C₆)alkylamino, phenyl, carboxy, (C₁-C₆)alkoxy,
(C₁-C₆)alkoxy(thio)carbonyl, hydrogeno(thio)carbonyl, sulfonato R—O—S(O)₂— or R—S(O)₂—O—, amide RR′N—C(O)— or R—C(O)—N(R′)— or acyl R—C(O)—, or ammonium RR′R″N⁺— groups with R, R′ and R″, which may be identical or different, representing a hydrogen atom or a (C₁-C₄)alkyl group.

3. The composition as claimed in either one of the preceding claims, characterized in that the photodimerizable group(s) is (are) chosen from the monovalent radicals of the following compounds:
stilbene,
styrylpyridinium (stilbazolium) having the formula below, and the geometric isomers thereof: 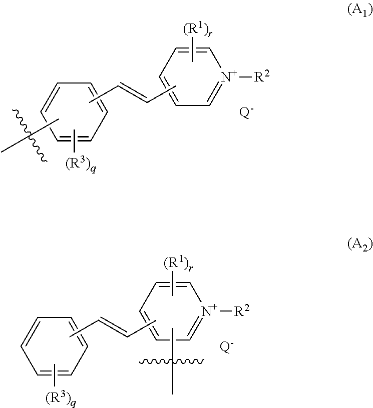
Where
R¹ and R³, which may be identical or different, represent a halogen atom or a (C₁-C₆)alkyl group; or alternatively two contiguous groups R¹ or R³ form, together with the carbon atoms that bear them, a benzo group;
R² represents a hydrogen atom, a (C₁-C₆)alkyl group optionally substituted with one or more halogen atoms such as chlorine or hydroxyl; preferably, R² represents a (C₁-C₆)alkyl group such as methyl, ethyl or propyl;
q and r represent an integer between 0 and 4 inclusive; and
Q⁻ represents an anionic counterion preferably chosen from halide ions such as chlorides, bromides, iodides, perchlorates, tetrafluoroborates, methylsulfate, phosphates, sulfates, methanesulfonates, p-toluenesulfonate; 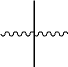
represents the bond that connects the part of the monovalent radical to the rest of the molecule being understood that the pendent group A₂ may be connected to the rest of the molecule via R²;
preferably the 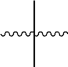
bond is found on the phenyl in the para position of the styryl group on A₁ or connected to the rest of the molecule via R² on A₂; preferentially the styryl group of A₁ and A₂ is found in the position para to the pyridinium group;
styrylazolium having the formula below, and the geometric isomers thereof: 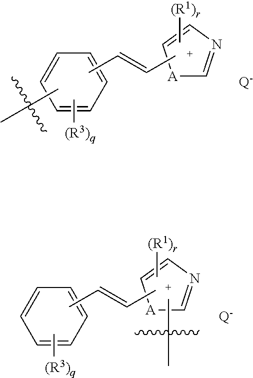
where:
A represents a sulfur atom, an oxygen atom, or a group NR² or C(R²)₂; and 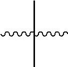
Q⁻, r, q, R¹, R², and R³ are as defined previously,
preferably the 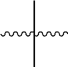
bond is found on the phenyl in the para position of the styryl group,
styrylpyrazine,
chalcone,
(thio)cinnamate and (thio)cinnamamide,
maleimide,
(thio)coumarin,
thymine,
uracil,
butadiene,
anthracene,
pyridone,
pyrrolizinone,
acridizinium salts,
furanone,
phenylbenzoxazole, and
derivatives thereof.

4. The composition as claimed in any one of the preceding claims, characterized in that the hydrophobic group(s) is (are) chosen from:
a (C₂-C₂₂)alkyl group, more preferentially a (C₃-C₁₆)alkyl group, preferably a (C₃-C₈)alkyl group.

5. The composition as claimed in any one of the preceding claims, characterized in that the photocrosslinkable polymer has a natural or synthetic backbone chosen from polysaccharides, poly(vinyl) polymers and polydiorganosiloxanes, preferably from polyvinyl alcohol and poly(vinyl acetate), which are preferably partially or totally hydrolyzed.

6. The composition as claimed in any one of the preceding claims, characterized in that the photocrosslinkable polymer(s) are chosen from the polymers comprising at least the units below: 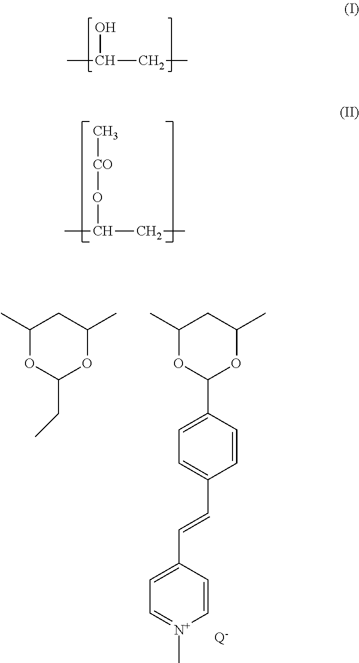
with Q⁻ representing an anionic counterion preferably chosen from halide ions such as chlorides, bromides, iodides, perchlorates, tetrafluoroborates, methylsulfate, phosphates, sulfates, methanesulfonates, p-toluenesulfonate, preferably mesylate CH₃OSO₃⁻.

7. The composition as claimed in any one of the preceding claims, characterized in that the total amount of the photocrosslinkable polymer(s) ranges from 0.01% to 40% by weight, preferably from 0.1% to 30% by weight, better still from 0.5% to 25% by weight and even better still from 1% to 10% by weight, relative to the total weight of the composition (C).

8. The composition as claimed in any one of the preceding claims, characterized in that the total content of pigments ranges from 0.05% to 20% by weight, preferably from 0.1% to 15% by weight and better still from 0.5% to 10% by weight, relative to the total weight of the composition.

9. The composition as claimed in any one of the preceding claims, characterized in that it also comprises at least one thickener.

10. The composition as claimed in any one of the preceding claims, characterized in that it comprises at least one silicone.

11. A process for the cosmetic treatment of, in particular for dyeing, keratin fibers such as the hair, wherein the composition (C) as defined in any one of claims 1 to 10 is applied to said fibers.

12. The process as claimed in claim 11, comprising a step of irradiating the composition on the keratin materials.

13. The use of the composition (C) as defined in any one of claims 1 to 10, for the cosmetic treatment of, in particular for dyeing, keratin fibers such as the hair.
